(19) United States
(12) Patent Application Publication
(10) Pub. No.: US 2025/0382261 A1
(43) Pub. Date: Dec. 18, 2025

(54) PENTAMIDINE ANALOGS

(71) Applicants: The Broad Institute, Inc., Cambridge, MA (US); MCMASTER UNIVERSITY, West Hamilton (CA); THE GENERAL HOSPITAL CORPORATION, Boston, MA (US)

(72) Inventors: Deborah HUNG, Cambridge, MA (US); Michael SERRANO-WU, Cambridge, MA (US); Katie LEE, Cambridge, MA (US); Brian HUBBARD, Cambridge, MA (US); Eric BROWN, Oakville (CA); Craig MACNAIR, Ancaster (CA); Maya FARHA, Ancaster (CA); Jean-Philippe COTE, Sherbrook (CA)

(73) Assignees: The Broad Institute, Inc., Cambridge, MA (US); MCMASTER UNIVERSITY, West Hamilton, ON (CA); THE GENERAL HOSPITAL CORPORATION, Boston, MA (US)

(21) Appl. No.: 18/653,324

(22) PCT Filed: Nov. 4, 2022
(86) PCT No.: PCT/CA2022/051635

§ 371 Date: May 2, 2024

Related U.S. Application Data
(60) Provisional application No. 63/276,473, filed on Nov. 5, 2021.

(51) Int. Cl. C07C 257/18; A61K 31/155; A61K 31/40; A61K 31/4178; A61K 31/444; A61K 31/451; A61K 31/7048; A61P 31/04; C07D 211/34; C07D 213/78; C07D 233/64; C07D 295/155

(52) U.S. Cl. CPC C07C 257/18; A61K 31/155; A61K 31/40; A61K 31/4178; A61K 31/444; A61K 31/451; A61K 31/7048; A61P 31/04; C07D 211/34; C07D 213/78; C07D 233/64; C07D 295/155; C07C 2601/08; C07C 2601/14; C07C 2601/18

(57) ABSTRACT

The present invention discloses novel pentamidine analogues such as pentamidine analogs having the general formula:

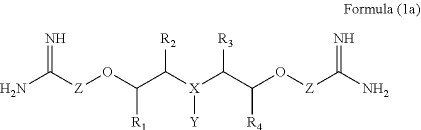

wherein:
X is C, N, or —CH—CH—,
Y is Y1 when X is N, and Y is Y1 and Y2 when X is C, or —CH—CH—,
Y1, or Y1 and Y2 independently, are selected from H, hydroxyl, lower alkyl, lower alkoxy, halogen, nitro, amino, cyano or thiol, wherein the lower alkyl or alkoxy is optionally substituted with one or more of hydroxyl, halogen, nitro, amino, cyano, thiol, or a 5- or 6-membered aromatic or non-aromatic ring, optionally substituted with one or more of hydroxyl, lower alkyl, lower alkoxy, halogen, nitro, amino, cyano or thiol, wherein Y1 is not H when X is N; or
Y1 is a 5- or 6-membered aromatic or non-aromatic ring, optionally substituted with one or more groups selected from hydroxyl, lower alkyl, lower alkoxy, halogen, nitro, amino, cyano, carboxy, or thiol, and Y2 is H, if present; or
Y1 and Y2 together with X form a 5- to 8-membered hydrocarbon ring, optionally substituted with one or more groups selected from hydroxyl, lower alkyl, lower alkoxy, halogen, nitro, amino, cyano, carboxyl, or thiol;
Z is phenyl, optionally substituted with one or more groups selected from hydroxyl, lower alkyl, lower alkoxy, halogen, nitro, amino, cyano, carboxyl, or thiol; and
R₁ to R₄ are each independently H, hydroxyl, halogen, lower alkyl or lower alkoxy;
   as well as related pentamidine analogs and their use to inhibit bacterial growth and treat bacterial infection.

### FIELD OF THE INVENTION

The present application generally relates to antibiotics, and more particularly relates to pentamidine analogs useful as antibiotics and antibiotic adjuvants.

### BACKGROUND

The spread of antibiotic resistance continues to outpace the development of new treatment options. This problem is particularly urgent for Gram-negative bacterial pathogens, for which no new class of clinically useful antibiotics has been discovered since the quinolones in 1967. Gram-negative bacteria are protected from noxious agents by an outer membrane (OM) barrier and efflux machinery. Unique to these organisms, the OM is an asymmetric lipid bilayer composed primarily of lipopolysaccharides (LPS) in the outer leaflet and phospholipids in the inner leaflet. The dense packing of LPS and negative charge of the OM provides a robust permeability barrier that is particularly effective in preventing small molecule accumulation within Gram-negative bacteria.

Antibiotics with activity against Gram-negative bacteria are almost entirely limited to molecules that pass through the OM via porins. Unfortunately, this entry route significantly restricts the range of physicochemical properties compatible with activity to only small and hydrophilic molecules. Because of this, readily identifiable enzymatic inhibitors of Gram-negative protein targets often fail to translate into whole-cell activity. Moreover, the intracellular targets for many large scaffold, Gram-positive-active antibiotics (macrolides, rifamycins, aminocoumarins) are conserved in Gram-negative bacteria, making OM permeation the only barrier to activity. Several approaches are currently in development to increase the intracellular concentration of these compounds in Gram-negative pathogens, including efflux inhibition, medicinal chemistry modifications, and chemical perturbation of the OM.

Disruption of the OM barrier facilitates the increased accumulation of many traditionally Gram-positive-active antibiotics in Gram-negative bacteria. Combining an OM-perturbing compound with a Gram-positive-active antibiotic has encouraging potential as an antibiotic approach, with several studies demonstrating activity in murine models of infection. This approach can overcome pre-existing resistance elements, reduce spontaneous resistance development and impair biofilm formation. An OM perturbing therapeutic would allow for the immediate use of many clinically available Gram-positive-active antibiotics against Gram-negative infections. Several groups have published proof of principle studies on the utility of this approach, identifying peptides, small molecules, and chelators that disrupt the OM. Most OM-active compounds are amphipathic molecules that are direct physical perturbants of LPS. Unfortunately, these compounds are limited by toxicity concerns due to their off-target disruption of host cell membranes. Achieving a therapeutic window between the disruption of bacterial and host cell membranes has proven difficult, with non-toxic derivatives often suffering from reduced activity.

Nevertheless, there is a compelling argument for the development of potent, non-toxic OM perturbants. Previous work has identified the cryptic OM-disrupting activity of the antiprotozoal drug pentamidine, which binds LPS and enhances sensitivity to a range of Gram-positive-active antibiotics (Stokes et al. 2017. Nature Microbiology, 2: 1-8). Combinations of pentamidine with Gram-positive-active antibiotics inhibit the growth of many Gram-negative species, including polymyxin-resistant Enterobacteriaceae and A. baumannii. However, the inherent toxicity of pentamidine remains a concern. Patients treated with pentamidine for Pneumocystis carinii pneumonia often develop nephrotoxicity, hypotension, hypoglycemia, and QT prolongation. The most troubling off-target effect is QT prolongation caused by a blockage of hERG trafficking and a reduction in the number of functional hERG channels.

Thus, it would be desirable to develop compounds having outer membrane disrupting activity for use to inhibit bacterial pathogens.

### SUMMARY OF THE INVENTION

Novel pentamidine analogs have been developed which exhibit outer membrane disrupting activity and reduced toxicity for use alone or in combination with antibiotics.

In one aspect, pentamidine analogs, and pharmaceutically acceptable salts, solvates and stereoisomers thereof, having the following general Formula (1a) are provided:

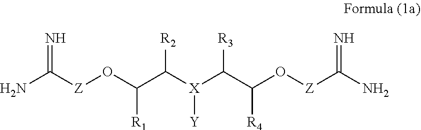

wherein:
X is C, N, or —CH—CH—,
Y is Y1 when X is N, and Y is Y1 and Y2 when X is C, or —CH—CH—,
Y1, or Y1 and Y2 independently, are selected from H, hydroxyl, lower alkyl, lower alkoxy, halogen, nitro, amino, cyano or thiol, wherein the lower alkyl or alkoxy is optionally substituted with one or more of hydroxyl, halogen, nitro, amino, cyano, thiol, or a 5- or 6-membered aromatic or non-aromatic ring, optionally substituted with one or more of hydroxyl, lower alkyl, lower alkoxy, halogen, nitro, amino, cyano or thiol, wherein Y1 is not H when X is N; or
Y1 is a 5- or 6-membered aromatic or non-aromatic ring, optionally substituted with one or more groups selected from hydroxyl, lower alkyl, lower alkoxy, halogen, nitro, amino, cyano, carboxy, or thiol, and Y2 is H, if present; or
Y1 and Y2 together with X form a 5- to 8-membered hydrocarbon ring, optionally substituted with one or more groups selected from hydroxyl, lower alkyl, lower alkoxy, halogen, nitro, amino, cyano, carboxyl, or thiol;
Z is phenyl, optionally substituted with one or more groups selected from hydroxyl, lower alkyl, lower alkoxy, halogen, nitro, amino, cyano, carboxyl, or thiol;
R₁ to R₄ are each independently H, hydroxyl, halogen, lower alkyl or lower alkoxy; with the proviso that the compound is not pentamidine.

In another aspect, a pentamidine analog, and pharmaceutically acceptable salts, solvates and stereoisomers thereof, having the general Formula (2) is provided:

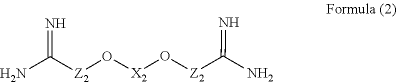

wherein
X₂ is a 5- to 8-membered hydrocarbon ring, optionally substituted with one or more groups selected from hydroxyl, lower alkyl, lower alkoxy, halogen, nitro, amino, cyano, carboxyl, or thiol, or
X₂ is C₁-C₃ alkyl chain, optionally substituted with one or two lower alkyl groups, wherein said lower alkyl groups may be joined to form a 5- to 8-membered hydrocarbon ring with one of C₁-C₃ of X₂, optionally substituted with one or more groups selected from hydroxyl, lower alkyl, lower alkoxy, halogen, nitro, amino, cyano, carboxyl, or thiol,
Z₂ is a 6-membered heterocyclic or non-heterocyclic ring, optionally substituted with one or more groups selected from hydroxyl, lower alkyl, lower alkoxy, halogen, nitro, amino, cyano, carboxyl, or thiol;
   with the proviso that the compound is not pentamidine.

In another aspect, a pentamidine analog, and pharmaceutically acceptable salts, solvates and stereoisomers thereof, is provided having the general structure of Formula (3):

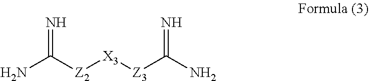

wherein
X₃ is -A-B-D-, wherein A is a 5- or 6-membered aromatic or non-aromatic heterocyclic or hydrocarbon ring, optionally substituted with one or more groups selected from hydroxyl, lower alkyl, lower alkoxy, halogen, nitro, amino, cyano, carboxy, or thiol, B is a lower alkyl chain and D is O, N or S; and
Z₂ is a 6-membered heterocyclic or non-heterocyclic ring, optionally substituted with one or more groups selected from hydroxyl, lower alkyl, lower alkoxy, halogen, nitro, amino, cyano, carboxyl, or thiol.

In a further aspect, a composition comprising a pentamidine analog having the general Formula (1a), Formula (2) or Formula (3), optionally in combination with an anti-bacterial compound is provided.

In a further aspect, a method of inhibiting bacterial growth is provided comprising administering to bacterial cells a pentamidine analog, or pharmaceutically acceptable salt, solvate or stereoisomer thereof, having the general Formula (1b):

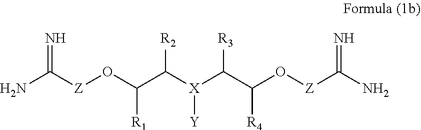

wherein:
X is C, N, or —CH—CH—,
Y is Y1 when X is N, and Y is Y1 and Y2 when X is C, or —CH—CH—,
Y1, or Y1 and Y2 independently, are selected from H, hydroxyl, lower alkyl, lower alkoxy, halogen, nitro, amino, cyano or thiol, wherein the lower alkyl or alkoxy is optionally substituted with one or more of hydroxyl, halogen, nitro, amino, cyano, thiol, or a 5- or 6-membered aromatic or non-aromatic ring, optionally substituted with one or more of hydroxyl, lower alkyl, lower alkoxy, halogen, nitro, amino, cyano or thiol; or
Y1 is a 5- or 6-membered aromatic or non-aromatic ring, optionally substituted with one or more groups selected from hydroxyl, lower alkyl, lower alkoxy, halogen, nitro, amino, cyano, carboxy, or thiol, and Y2 is H, if present; or
Y1 and Y2 together with X form a 5- to 8-membered hydrocarbon ring, optionally substituted with one or more groups selected from hydroxyl, lower alkyl, lower alkoxy, halogen, nitro, amino, cyano, carboxyl, or thiol;
Z is a 6-membered heterocyclic or non-heterocyclic ring, optionally substituted with one or more groups selected from hydroxyl, lower alkyl, lower alkoxy, halogen, nitro, amino, cyano, carboxyl, or thiol;
R₁ to R₄ are each independently H, hydroxyl, halogen, lower alkyl or lower alkoxy;
   with the proviso that the compound is not pentamidine.

In another aspect, a method of inhibiting bacterial growth is provided comprising administering to bacterial cells a pentamidine analog having the general Formula (1b), Formula (2) or Formula (3).

In another aspect, a method of treating a bacterial infection in a mammal is provided comprising administering to the mammal a composition comprising a pentamidine analog having the general Formula (1b), Formula (2) or Formula (3), optionally in combination with an anti-bacterial agent.

These and other aspects of the invention will become apparent by reference to the detailed description and figures described below.

### BRIEF DESCRIPTION OF THE FIGURES

FIG. 1. Pentamidine and analogs potentiate novobiocin in A. baumannii. a) Checkerboard assay showing dose-dependent potentiation of novobiocin by pentamidine against A. baumannii. Dark regions represent higher cell density. Data are representative of at least two biological replicates. b) Scatter plot of compound synergy (FICI) with novobiocin against A. baumannii and lipophilicity (c Log P). Analog FICI correlates with lipophilicity with r²=0.41 (simple regression, p<0.001). c) Scatter plot of FICI calculated as described in panel b and cytotoxicity (HEK293 AC₅₀) determined from a 7-point dose-response curve. Analogs P35 and P36 demonstrate improved potency and reduced toxicity. Pentamidine is highlighted in red and compounds P35 and P36 in blue.

FIG. 2. P35 potentiates novobiocin in a systemic animal infection model. a) P35-dependent potentiation of novobiocin in a systemic A. baumannii murine infection model. Animals were infected intraperitoneally with ˜2×10⁶ CFU, and treatments were administered 2 h post-infection. Treatment groups (n=5) included vehicle control (blue), 50 mg/kg novobiocin (red), 10 mg/kg P35 (green), 10 mg/kg P36 (black), 10 mg/kg pentamidine (brown) the combination of 10 mg/kg P35 and 50 mg/kg novobiocin (purple), 10 mg/kg pentamidine and 50 mg/kg novobiocin (orange), 10 mg/kg P36 and 50 mg/kg novobiocin (yellow). Clinical endpoint was defined using a body condition score, and the experimental endpoint was defined as 5 days post-infection. b) Using the same infection model and dosing as described for panel a, experimental endpoint was defined as 6 h post-infection. Animals (n=4) were euthanized, and colony-forming units (CFU/mL) within the blood were enumerated. Horizontal lines represent the geometric mean of bacterial load in each treatment group. The combination of P35 and novobiocin resulted in a significant reduction in CFU/mL compared to pentamidine and novobiocin (p<0.05, Mann-Whitney U-test).

FIG. 3. P35 potentiates Gram-positive antibiotics through a similar mechanism as pentamidine. a) Heat map showing antimicrobials potentiated (reduction in MIC>4-fold, green) or unaffected (reduction in MIC≤4-fold, black) by pentamidine (50 μg/mL) and P35 (50 μg/mL). b) Checkerboard assay showing dose-dependent potentiation of novobiocin by P35 against A. baumannii. c) Checkerboard as described in panel b with the addition of 20 mM Mg²⁺ to the growth media. d) Dose-dependent potentiation of novobiocin by P35 against K. pneumoniae. e) P35-dependent potentiation of novobiocin against mcr-1 expressing K. pneumoniae. Dark regions represent higher cell density. Data is representative of at least two biological replicates.

FIG. 4. P35 demonstrates improved cytotoxicity and reduced hERG channel inhibition. a) Ratio of HepG2 viability after 72 h incubation with increasing concentrations of pentamidine and P35 compared to a vehicle control. Data represent the mean viability at each concentration (n=3) plus or minus standard deviation. P35 has an AC₅₀ of 14.5 μM. Pentamidine inhibited cell viability at the lowest concentration tested (AC₅₀<0.293 μM). b) Hemolysis of human red blood cells in the presence of pentamidine (100 μm), P35 (100 μm) or Triton-X-100 control (0.1% and 1%). c) Pharmacokinetic analysis of pentamidine and P35. The mean plasma concentration and standard deviation after a single intravenous injection of 1 mg/kg P35 and pentamidine (n=3 per time point). d) Microelectrode Array (MEA) traces at baseline, 1 h and 48 h after exposure to DMSO, pentamidine (3.2 μM) or P35 (3.2 μM). MEA traces are representative of 13 average beats at each time point for pentamidine and 14 average beats for DMSO and P35. Pentamidine caused an increase in the beat period and field potential duration (FPD) due to hERG trafficking inhibition. P35 has only minor effects on the beat rate and FPD.

FIG. 5. Potency of potentiation correlates with LPS binding and is conserved between A. baumannii and K. pneumoniae. a) Scatter plot of compound synergy in A. baumannii (FICI) with novobiocin and LPS binding affinity as determined by BODIPY displacement (EC₅₀). FICI correlates with LPS binding r²=0.82. b,c,d) Scatter plots of analog activity (FICI) in b) A. baumannii and K. pneumoniae (r²=0.97), c) A. baumannii and P. aeruginosa (r²=0.45), and d) K. pneumoniae and P. aeruginosa (r²=0.46). Correlation is calculated using simple regression, p<0.001. d) Checkerboard assay showing suppression of dose-dependent potentiation of novobiocin by pentamidine against A. baumannii with the addition of 20 mM Mg²⁺ to the growth media. Dark regions represent higher cell density. Data is representative of at least 2 biological replicates.

### DETAILED DESCRIPTION OF THE INVENTION

Pentamidine analogs, and pharmaceutically acceptable salts thereof, for use to inhibit bacteria are provided, as well as pharmaceutically acceptable salts, solvates and stereoisomers thereof. The analogs have the following general Formula (1b):

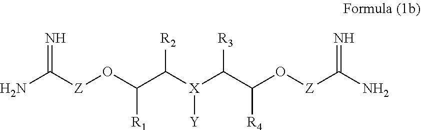

wherein:
X is C, N, or —CH—CH—,
Y is Y1 when X is N, and Y is Y1 and Y2 when X is C, or —CH—CH—,
Y1, or Y1 and Y2 independently, are selected from H, hydroxyl, lower alkyl, lower alkoxy, halogen, nitro, amino, cyano or thiol, wherein the lower alkyl or alkoxy is optionally substituted with one or more of hydroxyl, halogen, nitro, amino, cyano, thiol, or a 5- or 6-membered aromatic or non-aromatic ring, optionally substituted with one or more of hydroxyl, lower alkyl, lower alkoxy, halogen, nitro, amino, cyano or thiol; or
Y1 is a 5- or 6-membered aromatic or non-aromatic heterocyclic or hydrocarbon ring, optionally substituted with one or more groups selected from hydroxyl, lower alkyl, lower alkoxy, halogen, nitro, amino, cyano, carboxy, or thiol, and Y2 is H, if present; or
Y1 and Y2 together with X form a 5- to 8-membered hydrocarbon ring, optionally substituted with one or more groups selected from hydroxyl, lower alkyl, lower alkoxy, halogen, nitro, amino, cyano, carboxyl, or thiol;
Z is a 6-membered heterocyclic or non-heterocyclic ring, optionally substituted with one or more groups selected from hydroxyl, lower alkyl, lower alkoxy, halogen, nitro, amino, cyano, carboxyl, or thiol;
R₁ to R₄ are each independently H, hydroxyl, halogen, lower alkyl or lower alkoxy;
   with the proviso that the compound is not pentamidine.

The term “lower” as used herein with respect to alkyl and alkoxy groups refers to alkyl groups comprising 1-5 carbon atoms (e.g. C₁-C₅). The alkyl groups may be straight chain or branched alkyl groups.

The term “halogen” refers to fluorine, bromine or iodine.

Y1, and Y1 and Y2 (which may be the same or different), may be lower alkyl groups, optionally substituted. For example, Y1, and Y2, if present, may be selected from methyl, ethyl, propyl, isopropyl, butyl, isobutyl, etc. In embodiments, Y1 and Y2 are both methyl or ethyl. In other embodiments, Y1 is substituted with one or more halogen atoms, e.g. one or more of fluorine atoms, hydroxyl groups, nitro groups, amino groups and the like.

Y1 may be 5- or 6-membered aromatic or non-aromatic heterocyclic or hydrocarbon ring. Suitable 5-membered rings include, but are not limited to, cyclopentane and heterocyclic rings such as furan, tetrahydrofuran, pyrrolidine, pyrroline, pyrrole, pyrazolidine, imidazolidine, pyrazoline, imidazoline, pyrazole, imidazole, dioxolane, tetrahydrothiophene, thiophene, oxazole, isoxazole, thiazole, isothiazole and oxothiolane. Suitable 6-membered rings include, but are not limited to, cyclohexane and phenyl ring, as well as heterocyclic rings such as piperidine, pyridine, piperazine, pyrimidine, pyridazine, pyrazine, thiane, thiopyran, dithiane, morpholine, an oxazine, thiomorpholine and thiazine. In some embodiments, Y1 is phenyl.

Y1 and Y2 together with X may form a 5- to 8-membered ring. The ring may be saturated or unsaturated. Exemplary 5- to 6-membered rings include rings as described above. Additional suitable rings include cycloheptane, azepane, azepine, oxepane, oxepine, thiepane, thiepine, 3, 4, 5, 6-tetrahydro-2H-azepine, cyclooctane, azocane, thiocane and azocine. In some embodiments, Y1 and Y2 together with X form a hydrocarbon ring such as cyclopentane, cyclohexane, cycloheptane or cyclooctane.

Z is a 6-membered heterocyclic or non-heterocyclic ring. Preferably Z is aromatic. Exemplary rings include phenyl, pyridine, pyrimidine, pyridazine, pyrazine and 1,2,4-triazine. Z is optionally substituted with one or more groups such as hydroxyl, halogen, thiol, nitro and amino.

In one embodiment, the pentamidine analog comprises a structure in which Z is a phenyl group.

In another embodiment, the pentamidine analog comprises a structure in which Z is a phenyl group, optionally substituted, and in which X is carbon, optionally substituted, for example with one or more lower alkyl groups or other substituents, or X is C or —CHCH— and together with Y1 and Y2 forms a hydrocarbon ring which is optionally substituted.

In another embodiment, the pentamidine analog comprises a structure in which Z is a phenyl group, X is N, and Y1 is phenyl, optionally substituted, or lower alkyl, optionally substituted, for example with one or more halogen groups or a phenyl ring.

In a further embodiment, the pentamidine analogs are defined by Formula (1a), wherein:

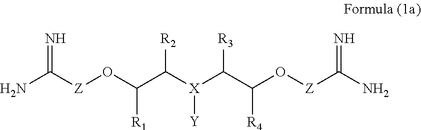

X is C, N, or —CH—CH—,
Y is Y1 when X is N, and Y is Y1 and Y2 when X is C, or —CH—CH—,
Y1, or Y1 and Y2 independently, are selected from H, hydroxyl, lower alkyl, lower alkoxy, halogen, nitro, amino, cyano or thiol, wherein the lower alkyl or alkoxy is optionally substituted with one or more of hydroxyl, halogen, nitro, amino, cyano, thiol, or a 5- or 6-membered aromatic or non-aromatic ring, optionally substituted with one or more of hydroxyl, lower alkyl, lower alkoxy, halogen, nitro, amino, cyano or thiol, wherein Y1 is not H when X is N; or
Y1 is a 5- or 6-membered aromatic or non-aromatic ring, optionally substituted with one or more groups selected from hydroxyl, lower alkyl, lower alkoxy, halogen, nitro, amino, cyano, carboxy, or thiol, and Y2 is H, if present; or
Y1 and Y2 together with X form a 5- to 8-membered hydrocarbon ring, optionally substituted with one or more groups selected from hydroxyl, lower alkyl, lower alkoxy, halogen, nitro, amino, cyano, carboxyl, or thiol;
Z is phenyl, optionally substituted with one or more groups selected from hydroxyl, lower alkyl, lower alkoxy, halogen, nitro, amino, cyano, carboxyl, or thiol;
R₁ to R₄ are each independently H, hydroxyl, halogen, lower alkyl or lower alkoxy;
   with the proviso that the compound is not pentamidine.

In another embodiment, the pentamidine analog has the general structure of Formula (2):

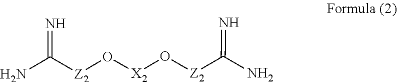

wherein
X₂ is a 5- to 8-membered hydrocarbon ring, optionally substituted with one or more groups selected from hydroxyl, lower alkyl, lower alkoxy, halogen, nitro, amino, cyano, carboxyl, or thiol, or
X₂ is C₁-C₃ alkyl chain, optionally substituted with one or two lower alkyl groups, wherein said lower alkyl groups may be joined to form a 5- to 8-membered hydrocarbon ring with one of C₁-C₃ of X₂, optionally substituted with one or more groups selected from hydroxyl, lower alkyl, lower alkoxy, halogen, nitro, amino, cyano, carboxyl, or thiol,
Z₂ is a 6-membered heterocyclic or non-heterocyclic ring, optionally substituted with one or more groups selected from hydroxyl, lower alkyl, lower alkoxy, halogen, nitro, amino, cyano, carboxyl, or thiol;
   with the proviso that the compound is not pentamidine.

In one embodiment, Z₂ is phenyl and X₂ is cyclohexyl.

In another embodiment, Z₂ is phenyl and X₂ is a C₃ alkyl chain in which C₂ is substituted with one or two lower alkyl groups which join to form cyclohexyl with C₂.

In another embodiment, the pentamidine analog has the general structure of Formula (3):

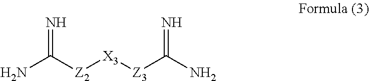

wherein
X₃ is -A-B-D- wherein A is a 5- or 6-membered aromatic or non-aromatic heterocyclic or hydrocarbon ring, optionally substituted with one or more groups selected from hydroxyl, lower alkyl, lower alkoxy, halogen, nitro, amino, cyano, carboxy, or thiol, B is a lower alkyl chain and D is O, N or S; and
Z₂ is a 6-membered heterocyclic or non-heterocyclic ring, optionally substituted with one or more groups selected from hydroxyl, lower alkyl, lower alkoxy, halogen, nitro, amino, cyano, carboxyl, or thiol.

A may be 5- or 6-membered aromatic or non-aromatic heterocyclic or hydrocarbon ring. Suitable 5-membered rings for A include, but are not limited to, cyclopentane and heterocyclic rings such as furan, tetrahydrofuran, pyrrolidine, pyrroline, pyrrole, pyrazolidine, imidazolidine, pyrazoline, imidazoline, pyrazole, imidazole, dioxolane, tetrahydrothiophene, thiophene, oxazole, isoxazole, thiazole, isothiazole and oxothiolane. Suitable 6-membered rings for A and for Z₂ include, but are not limited to, cyclohexane and phenyl ring, as well as heterocyclic rings such as piperidine, pyridine, piperazine, pyrimidine, pyridazine, pyrazine, thiane, thiopyran, dithiane, morpholine, an oxazine, thiomorpholine and thiazine.

In embodiments, A is tetrahydrofuran, pyrrolidine, piperidine or pyridine.

In embodiments, Z₂ is phenyl.

In one embodiment, Z₂ is phenyl, A is a piperadinyl ring, B is a lower alkyl chain and D is oxygen.

The present pentamidine analogs may be in the form of a pharmaceutically acceptable salt. The term “pharmaceutically acceptable salt” as used herein refers to acid or base addition salts of a pentamidine analog. Examples of inorganic acids that form acid addition salts with basic active agents include hydrochloric, hydrobromic, sulfuric, nitric and phosphoric acids, as well as acidic metal salts such as sodium monohydrogen orthophosphate and potassium hydrogen sulfate. Illustrative organic acids that form acid addition salts with basic active agents include mono-, di- and tricarboxylic acids. Illustrative of such organic acids are, for example, acetic, trifluoroacetic, propionic, glycolic, lactic, pyruvic, malonic, succinic, glutaric, fumaric, malic, oxalic, tartaric, citric, ascorbic, maleic, hydroxymaleic, benzoic, hydroxybenzoic, phenylacetic, cinnamic, mandelic, salicylic, 2-phenoxybenzoic, isethionic, p-toluenesulfonic acid and other sulfonic acids such as methanesulfonic acid, ethanesulfonic acid and 2-hydroxyethanesulfonic acid. Either the mono- or di-acid salts can be formed, and such salts can exist in either a hydrated, solvated or substantially anhydrous form.

Base addition salts are formed using inorganic bases such as lithium, sodium, potassium, calcium, magnesium or barium hydroxides, carbonates and bicarbonates, as well as ammonia. Illustrative organic bases that form base addition salts with acidic active agents include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, EGFRaine, ethylenediamine, glucosamine, methylglucamine, theobromine, purines, piperazine, piperidine, N-ethylpiperidine, polyamine resins, and the like. Illustrative organic bases are isopropylamine, diethylamine, ethanolamine, trimethylamine, dicyclohexylamine, choline, and caffeine.

In one embodiment, pentamidine analogs comprise structures that increase the lipophilicity of the analog so as to minimize or reduce mammalian cytoxicity, i.e. to possess lipophilicity wherein c Log P>0, for example, c Log P is >1, >1.5, or >2. c Log P is the logarithm of a compound's partition coefficient between n-octanol and water (log(c_{octanol}/c_{water}). Structures which contribute to the lipophilicity of the pentamidine analogs include the introduction of ring structures into the analog, e.g. introducing a ring at the central atom position of the analog (i.e. X in formula (1). Alternatively, a ring structure may be introduced as a substituent on the central atom to increase lipophilicity.

With respect to cytotoxicity, the pentamidine analog exhibits minimal mammalian cytotoxicity, and preferably is not toxic to mammalian cells. In one embodiment, the pentamidine analog is less toxic than pentamidine to mammalian cells. In other embodiments, the pentamidine analog exhibits an IC₅₀ that is >90 μg/ml, and preferably an IC₅₀ that is >100 μg/ml and more preferably, an IC₅₀ that is >90 μg/ml, >100 μg/mL, >150 μg/mL, or >200 μg/mL.

The present pentamidine analogs are effective alone as a monotherapy, i.e. not in combination with any other anti-bacterial agent, to inhibit undesirable bacterial growth. Thus, the pentamidine analog may be used to inhibit bacterial growth or administered to a mammal to treat a bacterial infection using a therapeutically effective dosage, i.e. a dosage effective to inhibit or at least reduce bacterial growth, for example, a dosage in the range of about 0.1-100 mg/kg. The terms “treat” or “treating” as used herein with respect to bacterial infection in a mammal refers to the inhibition of undesirable bacterial growth in a mammal, as well as the prevention of a bacterial infection in a mammal. The term “mammal” refers to both human and non-human mammals such as domestic animals, e.g. cats, dogs, horses, goats, sheep, pigs, cattle, rabbits, hamsters, guinea pigs, mice and rats, as well as wild animals.

Alternatively, the present pentamidine analogs are useful to inhibit bacterial growth in combination with an antibiotic agent. In this regard, the pentamidine analog is used to inhibit bacterial growth or is administered to a mammal to treat a bacterial infection at a dosage in the range of about 0.1-100 mg/kg in conjunction with a suitable dosage of an antibiotic agent. The phrase “in conjunction” refers to the administration of the pentamidine analog at the same time as the antibiotic agent, either in combination, or separately, as well as administration of the pentamidine analog at a time different from the administration of the antibiotic agent.

Without being limited to any particular theory, use of the pentamidine analogs in conjunction with an antibiotic agent may potentiate the antibacterial activity of the antibiotic agent. Thus, the combination of a pentamidine analog with a given antibiotic agent may result in an improvement in the activity of the antibiotic agent, e.g. wherein the minimum inhibitory concentration (MIC) of the antibiotic agent is reduced, to render a given antibiotic agent to be more effective, or to permit lower concentrations of a given antibacterial agent to be useful to inhibit bacterial growth. In some embodiments, the pentamidine analogs result in a synergistic effect when combined with a given antibiotic agent, i.e. wherein the MIC resulting from the combination is greater than the expected additive effect on MIC resulting from the combination of the pentamidine analog with the antibiotic agent. The effect of such a combination can be assessed on the basis of the fractional inhibitory concentration (FIC) index, which represents the sum of the FICs of the pentamidine analog and the antibiotic agent. The FIC is determined for each drug by dividing the MIC of each drug when used in combination by the MIC of each drug when used alone. A FIC index of <0.5 is indicative of synergy.

The present pentamidine analogs may be formulated for administration systemically, e.g. enterally or parenterally. Thus, administrable forms include, but are not limited to, oral (including sublingual and buccal), intravenous, intraperitoneal, subcutaneous, intramuscular, transepithelial, nasal, intrapulmonary, intrathecal, rectal, vaginal and intraocular. The analogs may also be formulated for administration topically or transdermally.

The pentamidine analogs, thus, may be combined with one or more pharmaceutically acceptable carriers, diluents or excipients. The expression “pharmaceutically acceptable” means acceptable for use in the pharmaceutical and veterinary arts, i.e. not being unacceptably toxic or otherwise unsuitable. Reference may be made to “Remington's: The Science and Practice of Pharmacy”, 21st Ed., Lippincott Williams & Wilkins, 2005, for guidance on drug formulations generally. The selection of adjuvant depends on the intended mode of administration of the composition. In one embodiment of the invention, the compounds are formulated for administration by infusion, or by injection either subcutaneously, intravenously, intrathecally, intraspinally or as part of an artificial matrix, and are accordingly utilized as aqueous solutions in sterile and pyrogen-free form and optionally buffered or made isotonic. Thus, a selected analog may be administered in distilled water or, more desirably, in saline, phosphate-buffered saline or 5% dextrose solution. Compositions for oral administration via tablet, capsule or suspension are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, anti-oxidants, preservatives, colouring agents and flavouring agents may also be present. In another embodiment, the composition may be formulated for application topically or transdermally as a cream, lotion or ointment. For such topical/transdermal applications, the composition may include an appropriate base such as a triglyceride base, and/or a carrier that facilitates absorption through the skin. Such creams, lotions and ointments may also contain a surface active agent and other cosmetic additives such as skin softeners and the like as well as fragrance. Aerosol formulations, for example, for nasal delivery, may also be prepared in which suitable propellant adjuvants are used. Compositions of the present invention may also be administered as a bolus, electuary, or paste.

Other adjuvants may also be added to the composition regardless of how it is to be administered, for example, preservatives, viscosity agents, buffering agents, binders, fillers, disintegrants, wetting agents, lubricants, glidants, coloring agents, dye-migration inhibitors, sweetening agents, and flavoring agents. Exemplary preservatives include benzalkonium chloride, chlorobutanol, thimerosal, benzyl alcohol, glycerin, methylparaben, propylparaben, benzoic acid, sodium benzoate or alcohol. Exemplary viscosity agents include methylcellulose, hyaluronic acid, propylene glycol, polycarbophil, mannitol or poloxamer 407. Exemplary buffering agents include citrate buffer, borate buffer, HCl/NaOH, distilled or purified water, or sodium chloride.

In other embodiments, the pentamidine analog may be combined with an adjuvant to potentiate or facilitate the activity of the pentamidine analog, either administered alone or in combination with an antibacterial agent. In this regard, the pentamidine analog may be combined with or administered in conjunction with a bicarbonate adjuvant. A bicarbonate adjuvant includes bicarbonate, i.e. HCO₃—, together with a cation. Thus, bicarbonate may be combined with an alkali metal cation to yield a bicarbonate adjuvant such as sodium bicarbonate, lithium bicarbonate or potassium bicarbonate; with an alkaline earth metal cation to yield a bicarbonate adjuvant such as magnesium bicarbonate or calcium bicarbonate; or the bicarbonate may be combined with other cationic groups to yield adjuvants such as ammonium bicarbonate or zinc bicarbonate. The dosage or amount of bicarbonate for use an adjuvant with a pentamidine analog is an amount that provides a physiological concentration of bicarbonate. In some embodiments, bicarbonate is used at a concentration of about 1 mM to about 900 mM, including about 25 nM, 50 nM, 100 to 800 nM, such as 200 mM, 300 mM, 400 mM, 500 mM, 600 mM and 700 mM. Alternatively, the bicarbonate is used in an amount of about 0.01 wt % to about 8.4 wt % of a composition.

The pentamidine analog may be administered in combination with an antibiotic agent effective against Gram negative bacteria or Gram positive bacteria, which is an obligate aerobe, obligate anaerobe or a facultative anaerobe. The bacteria, Gram negative or Gram positive, may be spiral-shaped, filamentous, pleomorphic, rectangular, sphere-shaped, or rod-shaped. In some embodiments, the bacteria to be treated is a species of Acinetobacter, Actinomyces, Aerococcus, Agrobacterium, Anaplasma, Azorhizobium, Azotobacter, Bacillus, Bacteroides, Bartonella, Bordetella, Borrelia, Brucella, Burkholderia, Calymmatobacterium, Campylobacter, Chlamydia, Chlamydophila, Clostridium, Corynebacterium, Coxiella, Ehrlichia, Enterobacter, Enterococcus, Escherichia, Francisella, Fusobacterium, Gardnerella, Haemophilus, Helicobacter, Klebsiella, Lactobacillus, Lactococcus, Legionella, Listeria, Methanobacterium, Microbacterium, Micrococcus, Moraxella, Mycobacterium, Mycoplasma, Neisseria, Pasteurella, Pediococcus, Peptostreptococcus, Porphyromonas, Prevotella, Pseudomonas, Rhizobium, Rickettsia, Rochalimaea, Rothia, Salmonella, Serratia, Shigella, Sarcina, Spirillum, Spirochaetes, Staphylococcus, Stenotrophomonas, Streptobacillus, Streptococcus, Tetragenococcus, Treponema, Vibrio, Viridans, Wolbachia or Yersinia.

In some embodiments, the bacteria to be treated is Acetobacter aurantius, Acinetobacter baumannii, Actinomyces israelii, Aerococcus viridans, Agrobacterium radiobacter, Agrobacterium tumefaciens, Anaplasma phagocytophilum, Azorhizobium caulinodans, Azotobacter vinelandii, Bacillus anthracis, Bacillus brevis, Bacillus cereus, Bacillus fusiformis, Bacillus lichenformis, Bacillus megaterium, Bacillus mycoides, Bacillus stearothermophilus, Bacillus subtilis, Bacillus thuringiensis, Bacteroides fragilis, Bacteroides gingivalis, Bacteroides melaninogenicus, Bartonella henselae, Bartonella quintana, Bordetella bronchiseptica, Bordetella pertussis, Borrelia burgdorferi. Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Campylobacter pylori, Chlamydia trachomatis, Chlamydophila pneumoniae, Chlamydophila psittaci, Clostridium botulinum, Clostridium difficile, Clostridium perfringens, Clostridium tetani, Corynebacterium diphtheriae, Corynebacterium fusiforme, CDC coryneform group G, Coxiella burnetii, Ehrlichia chaffeensis, Enterobacter cloacae, Enterococcus avium, Enterococcus durans, Enterococcus faecalis, Enterococcus faecium, Enterococcus gallinarum, Enterococcus maloratus, Escherichia coli, Francisella tularensis, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus bulgaricus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Neisseria gonorrhoeae, Neisseria meningitidis, Pasteurella multocida, Pasteurella tularensis, Peptostreptococcus, Porphyromonas gingivalis, Prevotella melaninogenica, Pseudomonas aeruginosa, Rhizobium radiobacter, Rickettsia prowazekii, Rickettsia psittaci, Rickettsia quintana, Rickettsia rickettsii, Rickettsia trachomas, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus (including MRSA or MSSA), Staphylococcus epidermidis, Stenotrophomonas maltophilia, Streptococcus agalactiae, Streptococcus avium, Streptococcus bovis, Streptococcus cricetus, Streptococcus faceium, Streptococcus faecalis, Streptococcus fetus, Streptococcus gallinarum, Streptococcus lactis, Streptococcus mitior, Streptococcus mitis, Streptococcus mutans, Streptococcus oxalis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Viridans streptococci, Wolbachia, Yersinia enterocolitica, Yersinia pestis or Yersinia pseudotuberculosis.

The pentamidine analogs may be utilized with an antibiotic agent such as, but not limited to, a macrolide, an aminoglycoside, a peptide, a glycopeptide, a β-lactam antibiotic, a quinolone, a fluoroquinolone or a rifampin, or a pharmaceutically acceptable salt thereof.

Examples of antibiotic agents with which the pentamidine analogs may be used include chloramphenicol, dirithromycin, erythromycin, linezolid, bacitracin, fosfomycin, fosmidomycin, vancomycin, polymyxin B, ciprofloxacin, besifloxacin, enoxacin, nalidixic acid, norfloxacin, levofloxacin, moxifloxacin, pefloxin, novobiocin, rifampicin, trimethoprim, tetracycline, cephalosporin, penicillin, carboxypenicillin, ureidopenicillin, β-lactamase inhibitors such as clavulanic acid, sulbactam and tazobactam, sulfamethoxazole, or a pharmaceutically acceptable salt thereof. In some embodiments, the antibiotic agent is an aminoglycoside such as apramycin, gentamicin, kanamycin, neomycin, paromycin, spectinomycin, or a pharmaceutically acceptable salt thereof. In some embodiments, the antibiotic agent is Amikacin, Apramycin, Gentamicin, Kanamycin, Neomycin, Tobramycin, Paromomycin, Streptomycin, Spectinomycin, Ertapenem, Doripenem, Imipenem/Cilastatin, Meropenem, Teicoplanin, Telavancin, Clindamycin, Lincomycin, Lipopeptide, Daptomycin, Azithromycin, Clarithromycin, Roxithromycin, Tulathromycin, Troleandomycin, Telithromycin, Spiramycin, Aztreonam, Posizolid, Radezolid, Torezolid, Colistin (Polymyxin E), Gatifloxacin, Gemifloxacin, Lomefloxacin, Ofloxacin, Trovafloxacin, Grepafloxacin, Sparfloxacin, Temafloxacin, Mafenide, Sulfacetamide, Sulfadiazine, Silver sulfadiazine, Sulfadimethoxine, Sulfamethizole, Sulfanilimide, Sulfasalazine, Sulfisoxazole, Sulfonamidochrysoidine, Arsphenamine, Chloramphenicol, Fusidic acid, Metronidazole, Mupirocin, Platensimycin, Quinupristin/Dalfopristin, Thiamphenicol, Tigecycline, Tinidazole, Clofazimine, Dapsone, Capreomycin, Cycloserine, Ethambutol, Ethionamide, Isoniazid, Pyrazinamide, Rifabutin, Rifapentine, Streptomycin, Thiostrepton, Doxycycline, Nafcillin, Ampicillin, Oxacillin, or a pharmaceutically acceptable salt thereof.

The term “about” as used herein indicates that a value may be somewhat altered from a recited value without substantial loss of activity or effect. For example, the term “about” may refer to a variance from a recited value of up to about 10%, either above or below the recited value. Thus, “about” includes a variance of 1%, 2%, 3%, 4%, 5%, 6%, 7%, 8%, 9% or 10% from a recited value.

Embodiments of the invention are described by reference to the Example that follows which is not to be construed as limiting.

### Example

Pentamidine analogs were synthesized and tested for anti-bacterial activity using the following methods.

Chemical synthesis of analogs. Analogs of pentamidine were synthesized by QuXi AppTec according to standard literature procedures. c Log P was calculated by importing compound SMILES into OSIRIS DataWarrior (Sander et al. Journal of Chemical Information and Modeling, 2015. 55: 460-73).

Bacterial strains and culture conditions. The following bacterial strains were used within this study; A. baumannii (ATCC 17978), K. pneumoniae (ATCC 43816), K. pneumoniae (ATCC 43816, expressing pGDP2:mcr-1), P. aeruginosa (PA01). Bacterial growth was in cation-adjusted Mueller-Hinton broth (MHB) at 37° C., and inoculum was prepared according to CLSI protocol (CLSI 2015).

Potentiation assays. Checkerboard analyses were conducted with each drug serially diluted at eight concentrations to create an 8×8 matrix. At least two biological replicates were done for each combination, and the means were used for FIC calculation. The FIC for each drug was calculated by dividing the concentration of the drug in the presence of codrug in a combination for a well showing <10% growth by the MIC for that drug alone. The FIC index (FICI) is the sum of the two FICs, with an FIC index≤0.5 deemed synergistic. Fold reduction of MIC was determined by dividing the MIC of the antibiotic alone by its MIC in the treatment condition: pentamidine (50 μg/mL) and P35 (50 μg/mL).

BODIPY-Cadaverine LPS displacement assay. The BODIPY-cadaverine LPS displacement assay was performed as previously described (Wood et al. 2004. Combinatorial Chemistry & High Throughput Screening, 7: 239-49; Klobucar et al. 2021. ACS Chemical Biology, 16: 929-42). A solution of BODIPY-cadaverine (2.25 μM) and LPS from E. coli EH100 (5.25 μM) in Tris-HCl buffer (50 mM, pH 7.4) was prepared. 0.5 μL of compound and 49.5 μL of the Tris-HCl buffer with BODIPY-cadaverine and LPS solution were added to a Corning nonbinding surface black 384-well polystyrene plate. Fluorescence intensity was measured immediately with an excitation wavelength of 580 nm and an emission wavelength of 620 nm. EC₅₀ values were calculated using a three-parameter logistic curve in GraphPad Prism 9.

Cytotoxicity Assays. HEK293 (ATCC CRL 1573) cells were left to adhere for 18 h at 7.5×10³ cells per well in DMEM containing 10% FBS and 2 mM L-glutamine. Compound was added directly into the well. After 48 h of treatment Promega Cell Titer Glo 2.0® was added, shaken for 10 min, and luminescence read (Biotek Neo). AC₅₀ values were calculated using a four-parameter logistic curve in GraphPad Prism 9.

HepG2 primary cell viability, phospholipidosis, and nuclear size assays were conducted by Cyprotex Ltd. Hepatocytes were plated on 384-well tissue culture treated black-walled, clear bottom polystyrene plates. Cells were incubated with P35 or pentamidine at 2-fold serial dilutions (8-point dose-response, 0.293 uM to 150 uM, 3 replicates per concentration). After 72 h of incubation, cells were loaded with the relevant dye and antibodies and analyzed using an automated fluorescent cellular imager (ArrayScan®).

Hemolysis was performed by Cyprotex Ltd in triplicate. Pentamidine and P35 were incubated at 100 μM in human blood for 45 min at 37° C., followed by an absorbance reading at 540 nm.

Mouse infection models. Animal experiments were conducted according to guidelines set by the Canadian Council on Animal Care using protocols approved by the Animal Review Ethics Board at McMaster University under Animal Use Protocol #20-12-43. Before infection, mice were relocated at random from a housing cage to treatment or control cages. No animals were excluded from analyses, and blinding was considered unnecessary. Seven- to nine-week-old female C57BL/6 mice (Charles River) were infected intraperitoneally with ˜2×10⁶ CFU of A. baumannii ATCC 17978, with 5% porcine mucin (Sigma-Aldrich). Infections were allowed to establish for 2 h, and treatments were administered intraperitoneally. For the survival study, the clinical endpoint was determined using a five-point body condition score analyzing weight loss, decrease in body temperature, respiratory distress, hampered mobility and hunched posture. Experimental endpoint was defined as five days post-infection for mice not reaching clinical endpoint. For CFU/mL enumeration, animals were infected as described above and euthanized 6 h post-infection, and blood was collected into sterile 2 ml heparinized tubes (BD Scientific) serially diluted in PBS and plated onto solid LB. Plates were incubated overnight at 37° C., and colonies were quantified to determine bacterial load.

Pharmacokinetic analysis. Female C57BL/6 mice were injected intravenously with a single dose of P35 (1 mg/kg) or pentamidine (1 mg/kg) in water and showed no adverse effects. Plasma samples were taken from 3 mice per time point (5, 15, 30 min; 1, 2, 4, 6 and 8 h post-dose). An aliquot of 2 μL sample was protein precipitated with 60 μL methanol, the mixture was vortex-mixed well and centrifuged at 13000 rpm for 10 min at 4° C. 2 μL supernatant was injected for LC-MS/MS analysis. The mean plasma concentration and the standard deviation from all 3 animals within each time point were calculated. PK parameters were calculated with a non-compartmental analysis. The mean plasma concentrations from all 3 mice at each time point were used in the calculations.

Plasma protein binding. Protein binding of P35 in mouse/rat/dog/human plasma was determined using an equilibrium dialysis method. The fraction of compound unbound to proteins was calculated with LC-MS/MS. This assay was performed in duplicate by Cyprotex Ltd.

Thermodynamic solubility. P35 in solution at pH 7.4 was incubated at ambient temperature overnight with stirring using a vial roller system. The saturation of the solution was determined using HPLC-UV. This assay was performed in duplicate by Cyprotex Ltd.

P450 inhibition. An 8-point dose-response of P35 (0, 0.1, 0.25, 1, 2.5, 10, 25 μM) was tested against the following cytochrome P450 (CYP) isoforms CYP3A4, CYP2C9, CYP2D6, CYP1A2, and CYP2C19. Reduction in metabolite formation was quantified using LC-MS/MS (with the exception of ethoxyresorufin for CYP1A2) to calculate an IC₅₀ value. This assay was performed by Cyprotex Ltd.

hERG channel assays. All assays were performed by Cyprotex Ltd. using an eCiphrCardio assay. CDI iCell® cardiomyocytes are plated onto fibronectin coated 48 well microelectrode array plates at a density of 50,000 cells per well in 5 μl iCell® plating medium. After 2 h, 300 μl of maintenance medium is added to each well. The plate is incubated for 2 days, and then the medium is completely changed to 500 μl of maintenance medium. The cells are incubated for an additional 3 days until a stable beating phenotype is achieved and the cells are ready for drug treatment. The medium is changed the day before dosing. The cells were treated with vehicle (0.2% DMSO), quinidine (positive control), pentamidine, or P35 in duplicate. The activity of cells in a 48-well microelectrode array is recorded prior to treatment (baseline) and at 1- and 48-hours post-treatment using the Axion Biosystems Maestro MEA system. The recording conditions are at 37° C. with 5% CO₂ using the standard cardiac settings on the Axion Biosystems Maestro Axis software version 2.1. The results are analyzed with the Axion Biosystem software.

### Results

Pentamidine and analogs potentiate novobiocin in A. baumannii. Pentamidine consists of two aromatic amidines connected by a lipophilic 5-carbon linker (Table 1). Despite extensive medicinal chemistry efforts to improve pentamidine as an antifungal and antiprotozoal (Porcheddu, Giacomelli, and De Luca 2012. Current Medicinal Chemistry, 19: 5819-36), limited work has investigated pentamidine as an OM perturbant. Using a small set of rationally-designed analogs, the importance of hydrophobicity and the retention of both cationic amidines for maintaining synergy with Gram-positive-active antibiotics was previously identified. Herein, a diverse set of analogs to probe the impact of rigidity, conformational flexibility, lipophilicity, chirality, and charge on the potentiation of Gram-positive-active antibiotics have been designed.

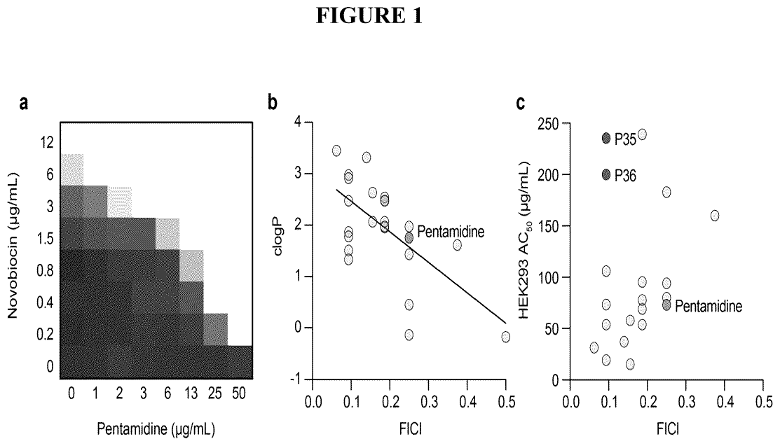

The pentamidine analogs were first tested for their ability to synergize with novobiocin to inhibit the growth of A. baumannii. Using checkerboard assays, the degree of potentiation by these analogs was quantified by calculating the fractional inhibitory concentration index (FICI), which was then compared to the FICI of 0.25 for pentamidine (FIG. 1a, Table 1). Lower FICI values represent a higher degree of potentiation, and all analog-novobiocin interactions with an FICI≤0.5 were deemed synergistic.

TABLE 1
Structure and activity of pentamidine analogs.

| | | LPS | HEK293T | | | | |
|---|---|---|---|---|---|---|---|
| | | Displacement | Cytotoxicity | FICI | | | |
| Analog | Structure | (EC₅₀, μg/mL) | (IC₅₀, μg/mL) | AB | KP | PA | cLog P |
| P02 | 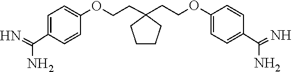 [SMILES: N=C(N)c1ccc(OCCC2(CCOc3ccc(C(=N)N)cc3)CCCC2)cc1] | 77.29 | 15.33 | 0.156 | 0.187 | 0.281 | 2.63 |
| P03 | 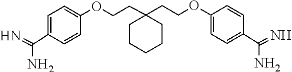 [SMILES: N=C(N)c1ccc(OCCC2(CCOc3ccc(C(=N)N)cc3)CCCCC2)cc1] | ND | ND | 0.094 | 0.078 | 0.625 | 2.98 |
| P04 | 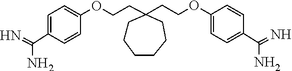 [SMILES: N=C(N)c1ccc(OCCC2(CCOc3ccc(C(=N)N)cc3)CCCCCC2)cc1] | 15.97 | 37.06 | 0.14 | 0.25 | 0.531 | 3.32 |
| P06 | 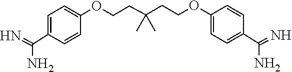 [SMILES: CC(C)(CCOc1ccc(C(=N)N)cc1)CCOc1ccc(C(=N)N)cc1] | 62.47 | 69.14 | 0.187 | 0.156 | 0.375 | 2.54 |
| P07 | 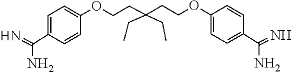 [SMILES: CCC(CC)(CCOc1ccc(C(=N)N)cc1)CCOc1ccc(C(=N)N)cc1] | 19.04 | 31.2 | 0.0625 | 0.156 | 1 | 3.45 |
| P08 | 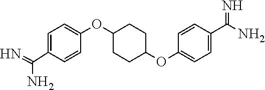 [SMILES: N=C(N)c1ccc(OC2CCC(Oc3ccc(C(=N)N)cc3)CC2)cc1] | 288.7 | >160 | 0.375 | 0.25 | 2 | 1.62 |
| P11 | 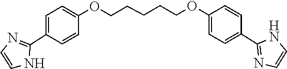 [SMILES: c1c[nH]c(-c2ccc(OCCCCCOc3ccc(-c4ncc[nH]4)cc3)cc2)n1] | >800 | 103.7 | 2 | 2 | 2 | 3.98 |
| P12 | 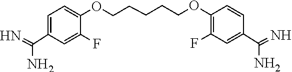 [SMILES: N=C(N)c1ccc(OCCCCCOc2ccc(C(=N)N)cc2F)c(F)c1] | 63.21 | 95.51 | 0.187 | 0.187 | 0.5 | 1.96 |
| P15 | 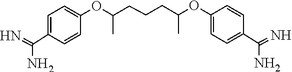 [SMILES: CC(CCCC(C)Oc1ccc(C(=N)N)cc1)Oc1ccc(C(=N)N)cc1] | ND | ND | 0.1875 | 0.25 | 0.281 | 2.47 |
| P15A | 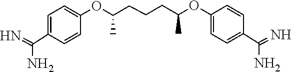 [SMILES: C[C@@H](CCC[C@H](C)Oc1ccc(C(=N)N)cc1)Oc1ccc(C(=N)N)cc1] | 118.2 | 106.1 | 0.094 | 0.25 | 0.5 | 2.47 |
| P15B | 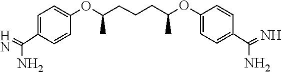 [SMILES: C[C@H](CCC[C@H](C)Oc1ccc(C(=N)N)cc1)Oc1ccc(C(=N)N)cc1] | 132.5 | 239.2 | 0.187 | 0.187 | 0.187 | 2.47 |
| P15C | 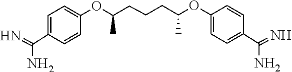 [SMILES: C[C@H](CCC[C@@H](C)Oc1ccc(C(=N)N)cc1)Oc1ccc(C(=N)N)cc1] | 148.8 | ND | 0.187 | 0.25 | 0.375 | 2.47 |
| P16 | 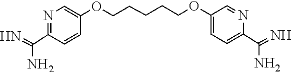 [SMILES: N=C(N)c1ccc(OCCCCCOc2ccc(C(=N)N)nc2)cn1] | 60.19 | 182.8 | 0.25 | 0.375 | 1 | −0.14 |
| P18 | 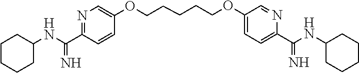 [SMILES: N=C(NC1CCCCC1)c1ccc(OCCCCCOc2ccc(C(=N)NC3CCCCC3)nc2)cn1] | 218.8 | 22.25 | 0.817 | 1 | 2 | 3.68 |
| P19 | 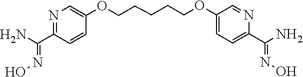 [SMILES: N/C(=N\O)c1ccc(OCCCCCOc2ccc(/C(N)=N/O)nc2)cn1] | >800 | 135 | 2 | 2 | 2 | 1.90 |
| P20 | 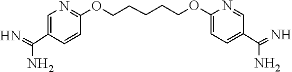 [SMILES: N=C(N)c1ccc(OCCCCCOc2ccc(C(=N)N)cn2)nc1] | ND | >320 | 0.25 | 0.25 | 2 | 0.46 |
| P21 | 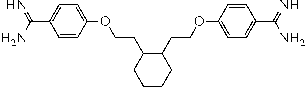 [SMILES: N=C(N)c1ccc(OCCC2CCCCC2CCOc2ccc(C(=N)N)cc2)cc1] | 13.08 | 19.18 | 0.094 | 0.046 | 1 | 2.91 |
| P22R | 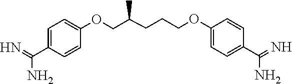 [SMILES: C[C@@H](CCCOc1ccc(C(=N)N)cc1)COc1ccc(C(=N)N)cc1] | 26.02 | 94.17 | 0.25 | 0.25 | 0.3125 | 1.97 |
| P22S | 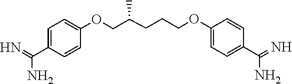 [SMILES: C[C@H](CCCOc1ccc(C(=N)N)cc1)COc1ccc(C(=N)N)cc1] | 59.08 | 53.73 | 0.187 | 0.25 | 0.375 | 1.97 |
| P24A | 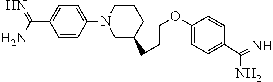 [SMILES: N=C(N)c1ccc(OCCC[C@@H]2CCCN(c3ccc(C(=N)N)cc3)C2)cc1] | 91.74 | 77.94 | 0.187 | 0.187 | 0.375 | 2.07 |
| P24B | 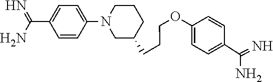 [SMILES: N=C(N)c1ccc(OCCC[C@H]2CCCN(c3ccc(C(=N)N)cc3)C2)cc1] | 149.5 | 57.91 | 0.156 | 0.187 | 0.75 | 2.07 |
| P30 | 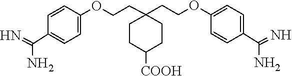 [SMILES: N=C(N)c1ccc(OCCC2(CCOc3ccc(C(=N)N)cc3)CCC(C(=O)O)CC2)cc1] | 119.4 | 1104 | 0.625 | 1 | 2 | 3.13 |
| P31 | 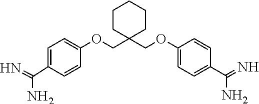 [SMILES: N=C(N)c1ccc(OCC2(COc3ccc(C(=N)N)cc3)CCCCC2)cc1] | 43.82 | 73.55 | 0.094 | 0.0937 | 0.187 | 1.87 |
| P31A | 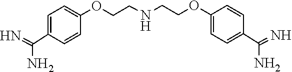 [SMILES: N=C(N)c1ccc(OCCNCCOc2ccc(C(=N)N)cc2)cc1] | 497.8 | >640 | 0.5 | ND | 2 | −0.18 |
| P34 | 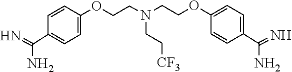 [SMILES: N=C(N)c1ccc(OCCN(CCOc2ccc(C(=N)N)cc2)CCC(F)(F)F)cc1] | 134.3 | >80 | 0.25 | ND | 0.375 | 1.44 |
| P35 | 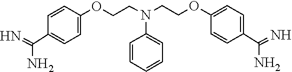 [SMILES: N=C(N)c1ccc(OCCN(CCOc2ccc(C(=N)N)cc2)c2ccccc2)cc1] | 87.21 | 235.4 | 0.094 | 0.12 | 0.3125 | 1.78 |
| P36 | 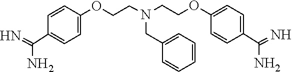 [SMILES: N=C(N)c1ccc(OCCN(CCOc2ccc(C(=N)N)cc2)Cc2ccccc2)cc1] | 165.9 | 200 | 0.094 | ND | 0.5 | 1.51 |
| P37 | 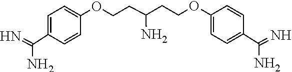 [SMILES: N=C(N)c1ccc(OCCC(N)CCOc2ccc(C(=N)N)cc2)cc1] | ND | ND | 0.75 | ND | 2 | 0.33 |
| P38 | 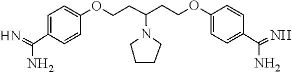 [SMILES: N=C(N)c1ccc(OCCC(CCOc2ccc(C(=N)N)cc2)N2CCCC2)cc1] | 202 | >80 | 0.75 | ND | 2 | 1.74 |
| P39 | 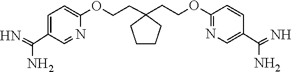 [SMILES: N=C(N)c1ccc(OCCC2(CCOc3ccc(C(=N)N)cn3)CCCC2)nc1] | 48.34 | 53.73 | 0.094 | ND | 0.3125 | 1.33 |
| Pent- amidine | 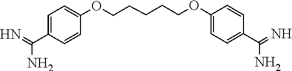 [SMILES: N=C(N)c1ccc(OCCCCCOc2ccc(C(=N)N)cc2)cc1] | 231 | 73.0 | 0.25 | 0.375 | 0.75 | 1.75 |

These structural analogs investigated several properties of pentamidine structure and their effect on novobiocin potentiation. Replacement of the two phenyl rings with pyridine heterocycles retained synergy with novobiocin (P16 and P20; FIC of 0.25, Table 1). The terminal amidine groups, however, were found to be essential—as amidines substituted with cyclohexyl (P18) or hydroxyl (P19) lacked novobiocin potentiating activity relative to P16, whilst the imidazole analog P11 also lost activity (FICI>0.5, Table 1). Flexibility within the linker region was found to be important for activity, as the rigid cyclohexyl linker (P8) decreased the FICI slightly to 0.375 (Table 1). Some degree of torsional gearing was favorable, however, as introduction of a gem-dimethyl group at the central carbon (P6) or methylation at the 1′ and 5′ carbon linker (P15) resulted in increased potency, FICI of 0.187 and 0.094, respectively (Table 1).

Long alkyl chains have been linked to increased mammalian cell membrane toxicity (Cook et al. 2019). Therefore, the potential to alter lipophilicity was considered in the linker region without increasing carbon chain length. Increasing lipophilicity through the addition of cyclopentane (P2) or cyclohexane (P3) at the central carbon increased potency compared to pentamidine (Table 1). In contrast, reducing lipophilicity by replacing the central carbon with nitrogen (P31A) reduced activity. Reintroducing lipophilicity into the P31A scaffold by addition of a benzene (P35) or benzyl group (P36) to the central nitrogen drastically increased potency (FICI of 0.094 for both molecules, Table 1). Overall, a rough correlation (r²=0.41, p<0.001, simple regression) was detected between lipophilicity (c Log P) and potency (FICI) (FIG. 1b). Indeed, 21 of 23 synergistic analogs (FICI≤0.5) were lipophilic (c Log P>0) (Table 1), suggesting that lipophilicity facilitates novobiocin potentiation.

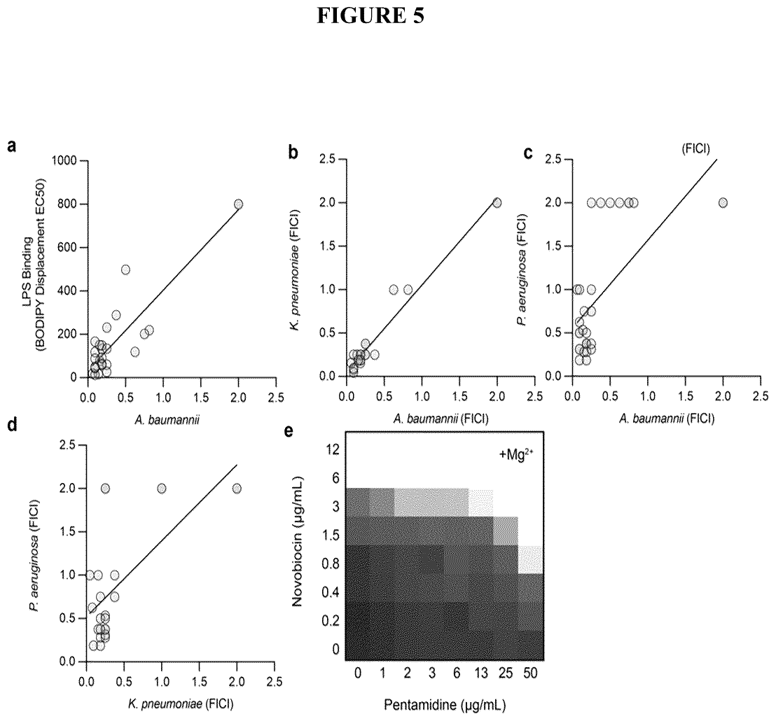

To investigate the influence of stereochemistry on potentiation activity, a series of chiral derivatives were designed. Minor changes were observed in potency between stereoisomers for all optically pure derivatives (P15A, P15B and P15C, P22R and P22S, P24A and P24B), all of which maintained synergy (FICI≤0.5, Table 1). Therefore, it was concluded that binding to a topologically-defined protein surface is unlikely to account for the biological activity of these analogs. Rather, binding of molecules to LPS was determined using a BODIPY displacement assay (FIG. 5). Pentamidine displayed a comparatively low affinity for LPS in this assay (EC₅₀ of 231 μg/mL), relative to the more potent LPS binding molecule colistin (EC₅₀ of 7.81 μg/mL). Affinity for LPS was highly correlated with potentiation (r²=0.82, p<0.001, simple regression). Indeed, the LPS binding affinity of the most potent analog, P07 (FICI 0.0625) was increased >10-fold (EC₅₀ of 19.04 μg/mL) compared to pentamidine. Additionally, analogs P11 and P19 were not synergistic with novobiocin (FICI>0.5), and both compounds had no detectable LPS binding.

Pentamidine sensitizes A. baumannii and Enterobacteriaceae species to Gram-positive-active antibiotics but lacks activity in P. aeruginosa (Stokes et al. 2017). To probe the spectrum of activity of the present analogs beyond A. baumannii, their interactions with novobiocin were assessed against the high-priority Gram-negative pathogens Klebsiella pneumoniae and P. aeruginosa (FIG. 5). FICI values between A. baumannii and K. pneumoniae were highly correlated (r²=0.92, p<0.001, simple regression). However, synergy in P. aeruginosa was less well correlated with A. baumannii or K. pneumoniae (r²=0.45 and 0.46 respectively, p<0.001, simple regression). Only 13 of the 23 analogs synergistic in A. baumannii demonstrated any synergy in P. aeruginosa, highlighting the difficulty in developing OM perturbants for this bacterial species. As A. baumannii was highly susceptible to potentiation, analogs were prioritized by their activity against this pathogen.

Due to the reported nephrotoxicity of pentamidine, the set of analogs was interrogated for host cell toxicity using embryonic HEK293 kidney cells. Focusing on only the analogs that retained synergistic activity with novobiocin (FICI≤0.5), half-maximal activity (AC₅₀) values were calculated against HEK293 cells using a CellTiter-Glo® based assay to approximate cell viability. As expected, pentamidine was cytotoxic in the assay with an AC₅₀ of 73 μg/mL. When analog potency was directly compared in A. baumannii (FICI) to cytotoxicity (AC₅₀) (FIG. 1c), the majority of compounds clustered with pentamidine along the toxicity axis. However, the two analogs P35 and P36, had both increased potency and decreased cytotoxicity relative to pentamidine. Specifically, the FICI value for both P35 and P36 was 0.094 against A. baumannii, while their AC₅₀ values were 235 μg/mL and 200 μg/mL, respectively. Both analogs are derivatives of the P31A scaffold, with a central carbon to nitrogen substitution in the linker region of pentamidine.

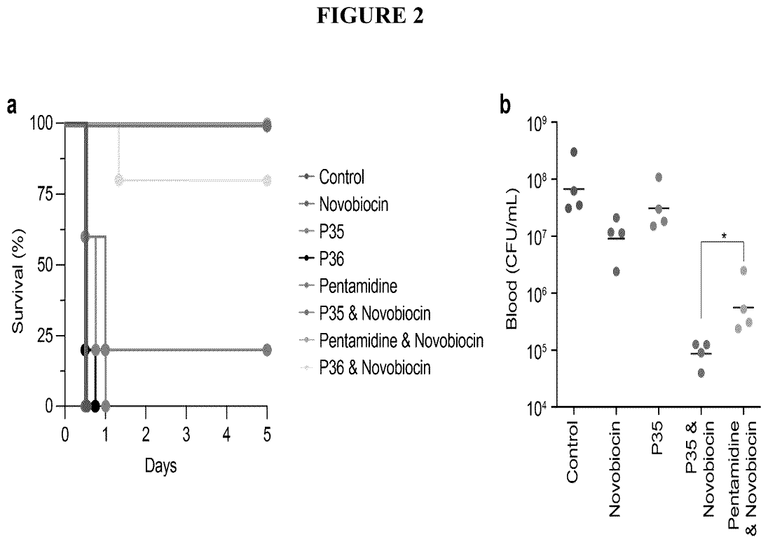

P35 potentiates novobiocin in a systemic animal infection model. Considering the reduced toxicity and increased potency of P35 and P36, the in vivo activity of these two analogs was investigated. Previous work reported the ability of pentamidine to rescue 100% of infected animals when administered in combination with novobiocin in a murine model of systemic A. baumannii infection (Stokes et al. 2017). This was directly compared to the activity of P35 and P36 in this model (all administered at 10 mg/kg) in combination with novobiocin (50 mg/kg). Animals treated with P35, P36 or novobiocin alone reached clinical endpoint within 24 h of infection (FIG. 2a). Aligning with previous work, pentamidine rescued 100% of animals when administered in combination with novobiocin. Encouragingly, P35 and P36 rescued 100% and 80% of animals, respectively, when combined with novobiocin.

Given the superior in vivo efficacy of P35 over P36, this molecule was the focus of further experiments. P35 was equivalent to pentamidine with respect to animal survival in this infection model (100% of animals rescued), so efficacy was more precisely compared by quantifying bacterial burden in the blood of infected animals. For this experiment the same A. baumannii infection model was used, however, endpoint was defined as 6 h post-infection, at which point blood was collected for CFU/mL enumeration (FIG. 2b). Treatment with P35 or novobiocin alone had no significant impact on blood CFU/mL compared to control-treated animals. The combination of P35 and novobiocin resulted in a 3.05-log₁₀ reduction in CFU/mL. Notably, this reduction in CFU/mL by P35 and novobiocin was significantly better than the 2.08-log₁₀ reduction observed with the combination of pentamidine and novobiocin (p<0.05, Mann-Whitney U-test).

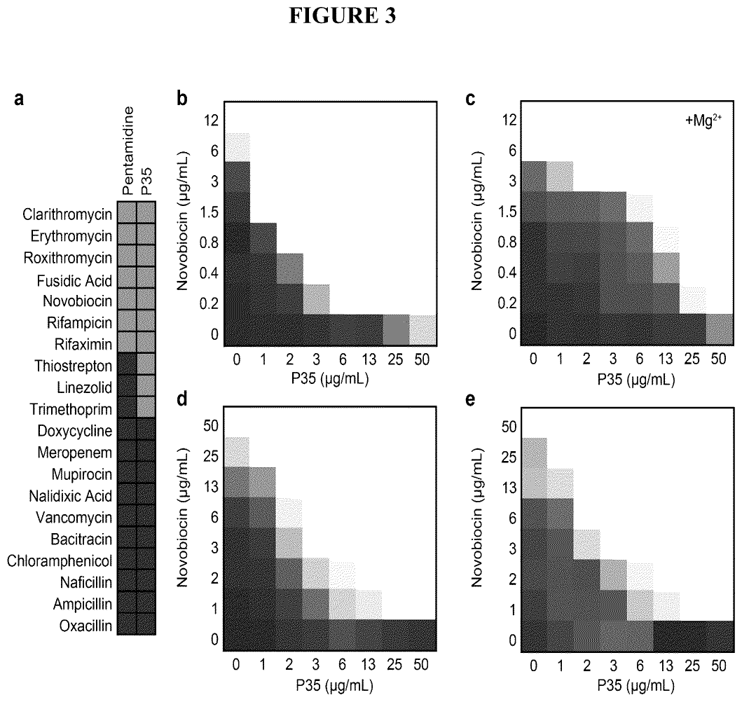

P35 potentiates Gram-positive antibiotics through a similar mechanism as pentamidine. To further investigate the potential of P35 as an OM perturbant, the spectrum of antibiotics potentiated by this analog were compared to those potentiated by pentamidine. A panel of 20 antibiotics were screened for potentiation with P35 and pentamidine at fixed concentrations (50 μg/mL) against A. baumannii (FIG. 3a, Table 2). Antibiotics were considered to be potentiated if their MIC was reduced >4-fold compared to a no-treatment control. Seven of the 20 antibiotics were potentiated by pentamidine, in agreement with the spectrum of activity reported against E. coli (MacNair and Brown 2020). Those that were potentiated in these assays consisted of traditionally Gram-positive antibiotics, particularly large and hydrophobic molecules. These results indicated that antibiotic potentiation was phenotypically similar between pentamidine and P35, with all 7 compounds potentiated by pentamidine also synergizing with P35. Further, P35 potentiated 3 additional antibiotics compared to pentamidine (thiostrepton, linezolid, trimethoprim). Notably, the fold-reduction in MIC with P35 was higher than pentamidine for all potentiated antibiotics, suggesting a general increase in potency.

TABLE 2
Fold-reduction values of antibiotics by
pentamidine and P35 in A. baumannii.

Average Fold-reduction in MIC

| | Pentamidine | P35 |
|---|---|---|
| Clarithromycin | 12 | 384 |
| Erythromycin | 8 | 32 |
| Roxithromycin | 10.67 | 341.33 |
| Fusidic Acid | 64 | 341.33 |
| Novobiocin | 64 | 512 |
| Rifampicin | 24 | 384 |
| Rifaximin | 32 | 256 |
| Thiostrepton | 1 | 170.67 |
| Linezolid | 4 | 8 |
| Trimethoprim | 2 | 5.33 |
| Doxycycline | 4 | 4 |
| Meropenem | 1 | 4 |
| Mupirocin | 1.33 | 4 |
| Nalidixic Acid | 4 | 4 |
| Vancomycin | 1.5 | 4 |
| Bacitracin | 1.6 | 2 |
| Chloramphenicol | 3.2 | 2 |
| Nafcillin | 0.75 | 1.5 |
| Ampicillin | 1 | 1 |
| Oxacillin | 1.33 | 1 |

The conserved spectrum of antibiotic potentiation and strong LPS binding of P35 suggests that its synergy with Gram-positive-active antibiotics involves a mechanism of action similar to pentamidine. OM disruption by pentamidine is predicted to proceed through direct interactions with lipid A, displacing cation bridging between LPS molecules. As such, the potentiation activity of pentamidine can be suppressed through the addition of exogenous Mg²⁺ in the growth media, and similar results were observed for P35 activity (FIG. 3b, FIG. 3c). The presence of 20 mM Mg²⁺ reduces the FICI of P35 from 0.094 to 0.56, consistent with P35 inducing OM disruption by binding LPS.

Despite interacting with LPS, pentamidine retains the ability to potentiate antibiotics in bacteria expressing the polymyxin-resistance gene mcr-1, which masks the negative charge of lipid A through the addition of a positively charged phosphoethanolamine group. With the increasing incidence of polymyxin-resistance in the clinic, the question of maintaining potentiation activity in these strains is an important one for the P35 compound. P35 and novobiocin are highly synergistic against K. pneumoniae (FICI=0.12) (FIG. 3d). Notably, this activity is unchanged (FICI=0.12) (FIG. 3e) against K. pneumoniae expressing mcr-1, suggesting that the activity of P35 is independent of phosphoethanolamine modifications to lipid A.

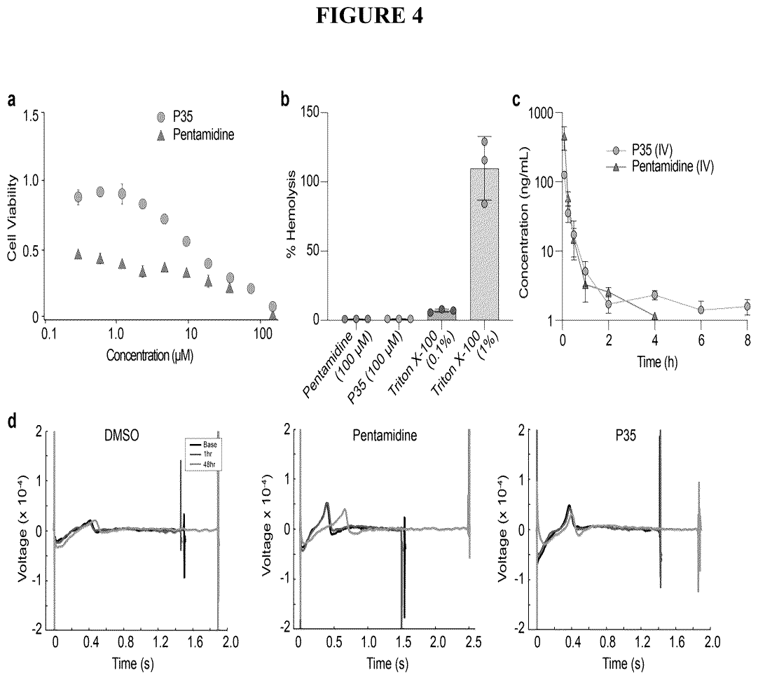

P35 demonstrates improved cytotoxicity and reduced hERG channel inhibition. To further investigate the potential of P35, additional toxicity, absorption, distribution, metabolism and excretion (ADME) assays were conducted. P35 demonstrated reduced mammalian cytotoxicity in HEK293 experiments. Using HepG2 cells, the impact of compound exposure on cell cycle arrest (FIG. 4a), nuclear size, and phospholipidosis after 72 h was assessed (Table 3). Pentamidine causes cell cycle arrest (AC₅₀<0.293 μM) likely at the G2 to M transition as cells display enlarged nuclei at all concentrations tested. P35 has a reduced impact on HepG2 cell cycle arrest (AC₅₀ of 14.5 μM, FIG. 4a) and nuclear size (Table 3), for which P35 has a minimum effective concentration (MEC) of 3.17. P35 demonstrated phospholipidosis AC₅₀=43.1 μM, while no response (>150 μM) was observed for pentamidine. Neither compound was hemolytic to human blood at 100 μM (FIG. 4b).

TABLE 3
HepG2 cell health parameters after 72 h incubation.

| | Nuclear Size | | | Phospholipidosis | | |
|---|---|---|---|---|---|---|
| | AC50 (μM) | MEC (μM) | MR (μM) | AC50 (μM) | MEC (μM) | MR (μM) |
| Pentamidine | 2.28 (NS) | <0.293 | 1.07 (19% | NR | NR | NR |
| | | | @ 0.293 | | | |
| | | | μM) | | | |
| P35 | 94.5 | 3.17 | 1.11 (27% | 43.1 | ↑ 27.4 | 6.20 (104% |
| | (r² 0.924) | | @ 18.8 μM) | (r² 0.965) | | @ 75 μM) |

Minimum effective concentration (MEC) that significantly crosses vehicle control threshold. Maximum response (MR) as a ratio of the control.
↑ Direction of response. No response observed (NR), fit not statistically significant (NS).

Pentamidine rapidly clears from plasma after injection and accumulates in organ tissues. To determine if the structural modification of P35 altered pharmacokinetics, mice were injected intravenously with 1 mg/kg of P35 or pentamidine and serum levels were monitored over eight hours (FIG. 4c, Table 4). Pentamidine concentrations were higher than P35 at all time points. However, both compounds were rapidly cleared with a half-life of 1.61 h and 1.96 h for P35 and pentamidine, respectively.

TABLE 4
PK parameters of pentamidine and P35.

| | Pentamidine (IV) | P35 (IV) |
|---|---|---|
| Rsq_adj | 0.976 | 0.504 |
| No. points used for T_{1/2} | 3.00 | 8.00 |
| C₀ (ng/mL) | 1264 | 238 |
| T_{1/2} (h) | 1.96 | 1.61 |
| Vdₛₛ (L/kg) | 3.45 | 33.4 |
| Cl (mL/min/kg) | 140 | 301 |
| Tₗₐₛₜ (h) | 4.00 | 8.00 |
| AUC₀₋ₗₐₛₜ (ng · h/mL) | 116 | 51.6 |
| AUC_{0-inf} (ng · h/mL) | 119 | 55.3 |
| MRT₀₋ₗₐₛₜ (h) | 0.229 | 1.24 |
| MRT_{0-inf} (h) | 0.411 | 1.85 |
| AUC_{Extra} (%) | 2.76 | 6.74 |
| AUMC_{Extra} (%) | 45.8 | 37.7 |

Mean residence time (MRT).

Additional ADME parameters, including solubility, P450 inhibition, and protein binding, were determined for P35 (Table 5). With a kinetic solubility of 182.4 μM, P35 is well suited for intravenous administration. Additionally, P35 does not demonstrate high levels of protein binding in mouse, rat, dog and human serum. P35 has no predicted drug-drug interactions, with a P450 inhibition AC₅₀ of 14.8 μM for CYP2D6 and >50 μM for all other isoforms tested.

TABLE 5
ADME parameters of P35.

| | P35 |
|---|---|
| Kinetic solubility | 182.4 |
| (μM @ pH 7.4) | |
| In vitro metabolic stability | 91.6/110.5/137.1/>145 |
| (t_{1/2}, min − m/r/d/h) | |
| P450 Inhibition | 2D6 IC₅₀ = 14.8 μM |
| CYP 3A4, 2C9, 2D6, 1A2, | 3A4, 2C9, 1A2, 2C19 IC₅₀ > 50 μM |
| 2C19 (IC₅₀, μM) | |
| Protein binding | 83.1/92.3/87.4/87.0 |
| (%, m/r/d/h) | |

Mouse (m), rat (r), dog (d), and human (h).

One of the most concerning side-effects of pentamidine use is QT prolongation due to inhibition of hERG channel trafficking. Neither pentamidine nor P35 directly inhibited the hERG channel 1 h after exposure (FIG. 4d). However, after 48 h, pentamidine at 3.2 μM was proarrhythmic, causing an increase in the beat period and field potentiation duration due to hERG trafficking inhibition. Notably, no change in QT prolongation was observed with P35 at 3.2 μM. Attempts at repeating this assay at 10 μM resulted in the loss of all detectable beating before completion of the 48 h incubation for P35 and pentamidine.

### DISCUSSION

An extensive medicinal chemistry effort led to the identification of the effective pentamidine analogs. For example, in one embodiment, compared to pentamidine, analog P35 potentiation was increased for a broad spectrum of antibiotics and the potentiation activity was retained in polymyxin-resistant bacteria. The combination of P35 with novobiocin had improved efficacy in a murine model of systemic A. baumannii infection, significantly reducing bacterial load when compared to pentamidine and novobiocin. The spectrum of antibiotic potentiation, ability to bind LPS, and suppression of novobiocin synergy with exogenous Mg²⁺ all suggest that P35 possesses a similar mechanism of action as pentamidine, involving direct interaction with the phosphates in lipid A to disrupt OM integrity. Notably, the improved potency of P35 did not increase mammalian cell toxicity. P35 had no hemolytic activity and exhibited reduced cytotoxicity against HEK293 and HepG2 cells compared to pentamidine. P35 also had a reduced impact on hERG channel trafficking relative to pentamidine, overcoming one of the biggest toxicity hurdles.

Thus, this study highlights the ability to separate OM-disrupting activity from host cell toxicity through a medicinal chemistry approach. The clinical implementation of pentamidine analog OM perturbants like P35 provide a means to sensitize Gram-negative pathogens to dozens of otherwise inactive antibiotics and provide a reprieve from the resistance crisis.

1. A pentamidine analog having the following general Formula (1a): 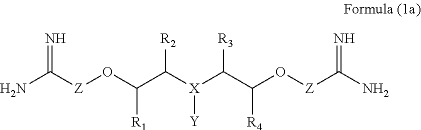
wherein:
X is C, N, or —CH—CH—,
Y is Y1 when X is N, and Y is Y1 and Y2 when X is C, or —CH—CH—,
Y1, or Y1 and Y2 independently, are selected from H, hydroxyl, lower alkyl, lower alkoxy, halogen, nitro, amino, cyano or thiol, wherein the lower alkyl or alkoxy is optionally substituted with one or more of hydroxyl, halogen, nitro, amino, cyano, thiol or a 5- or 6-membered aromatic or non-aromatic ring, optionally substituted with one or more of hydroxyl, lower alkyl, lower alkoxy, halogen, nitro, amino, cyano or thiol, wherein Y1 is not H when X is N; or
Y1 is a 5- or 6-membered aromatic or non-aromatic ring, optionally substituted with one or more groups selected from hydroxyl, lower alkyl, lower alkoxy, halogen, nitro, amino, cyano, carboxy, or thiol, and Y2 is H, if present; or
Y1 and Y2 together with X form a 5- to 8-membered hydrocarbon ring, optionally substituted with one or more groups selected from hydroxyl, lower alkyl, lower alkoxy, halogen, nitro, amino, cyano, carboxyl, or thiol;
Z is phenyl, optionally substituted with one or more groups selected from hydroxyl, lower alkyl, lower alkoxy, halogen, nitro, amino, cyano, carboxyl, or thiol;
R₁ to R₄ are each independently H, hydroxyl, halogen, lower alkyl or lower alkoxy;
with the proviso that the compound is not pentamidine.

2. The pentamidine analog as defined in claim 1, wherein Z is a phenyl group, X is N, and Y1 is phenyl, optionally substituted with hydroxyl, lower alkyl, lower alkoxy, halogen, nitro, amino, cyano or thiol, or Y1 is lower alkyl, optionally substituted with one or more halogen groups or a phenyl ring.

3. The pentamidine analog as defined in claim 1, wherein Z is a phenyl group substituted with a halogen and X is C.

4. The pentamidine analog as defined in claim 1, wherein Z is a phenyl group, X is C, and Y1 and Y2 are lower alkyl.

5. The pentamidine analog as defined in claim 1, wherein Z is a phenyl group, X is C, and one or more of R₁, R₂, R₃ and R₄ are lower alkyl.

6. The pentamidine analog as defined in claim 1, wherein Z is a phenyl group, X is C, and R₁ and R₄ are lower alkyl.

7. The pentamidine analog as defined in claim 1, wherein Z is a phenyl group, X is C, and Y1 and Y2 together with X form a 5- to 8-membered hydrocarbon ring.

8. The pentamidine analog as defined in claim 1, wherein X is N and Y1 is benzyl or a halogen-substituted lower alkyl group.

9. The pentamidine analog as defined in claim 1, wherein Z is a phenyl group optionally substituted with halogen, and X is C, wherein Y1 is H and Y2 is H, amino or a 5- or 6-membered aromatic or non-aromatic ring.

10. The pentamidine analog as defined in claim 1, wherein Z is a phenyl group, X is —CH—CH—, and Y1 and Y2 together with X form a 5- to 8-membered hydrocarbon ring.

11. A pentamidine analog having the general Formula (2): 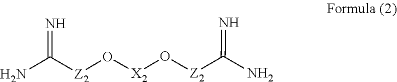
wherein
X₂ is a 5- to 8-membered hydrocarbon ring, optionally substituted with one or more groups selected from hydroxyl, lower alkyl, lower alkoxy, halogen, nitro, amino, cyano, carboxyl, or thiol, or
X₂ is C₁-C₃ alkyl chain, optionally substituted with one or two lower alkyl groups, wherein said lower alkyl groups may be joined to form a 5- to 8-membered hydrocarbon ring with one of C₁-C₃ of X₂, optionally substituted with one or more groups selected from hydroxyl, lower alkyl, lower alkoxy, halogen, nitro, amino, cyano, carboxyl, or thiol,
Z₂ is a 6-membered heterocyclic or non-heterocyclic ring, optionally substituted with one or more groups selected from hydroxyl, lower alkyl, lower alkoxy, halogen, nitro, amino, cyano, carboxyl, or thiol;
with the proviso that the compound is not pentamidine.

12. The pentamidine analog of claim 11, wherein Z₂ is phenyl.

13. The pentamidine analog of claim 11, wherein Z₂ is phenyl and X₂ is a C₃ alkyl chain in which C₂ is substituted with one or two lower alkyl groups which join to form cyclohexyl with C₂.

14. The pentamidine analog of claim 11, wherein Z₂ is a heterocyclic aromatic ring, X₂ a 5- to 8-membered hydrocarbon ring.

15. The pentamidine analog of claim 14, wherein Z₂ is a pyridine ring.

16. A pentamidine analog having the general structure of Formula (3): 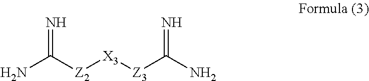
wherein
X₃ is -A-B-D-, wherein A is a 5- or 6-membered aromatic or non-aromatic heterocyclic or hydrocarbon ring, optionally substituted with one or more groups selected from hydroxyl, lower alkyl, lower alkoxy, halogen, nitro, amino, cyano, carboxy, or thiol, B is a lower alkyl chain and D is O, N or S; and
Z₂ is a 6-membered heterocyclic or non-heterocyclic ring, optionally substituted with one or more groups selected from hydroxyl, lower alkyl, lower alkoxy, halogen, nitro, amino, cyano, carboxyl, or thiol.

17. The pentamidine analog of claim 16, wherein Z₂ is phenyl, A is a piperadinyl ring, B is a lower alkyl chain and D is oxygen.

18. The pentamidine analog of claim 18, wherein X, X₂ or X₃ comprises a ring structure.

19. A composition comprising a pentamidine analog as defined in claim 1, in combination with a pharmaceutically acceptable carrier, adjuvant or excipient.

20. A method of treating a bacterial infection in a mammal comprising administering to the mammal a pentamidine analog as defined in claim 1, or having the general Formula (1b): 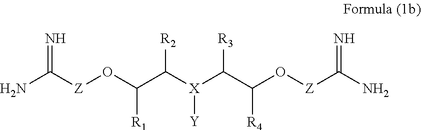
wherein:
X is C, N, or —CH—CH—,
Y is Y1 when X is N, and Y is Y1 and Y2 when X is C, or —CH—CH—,
Y1, or Y1 and Y2 independently, are selected from H, hydroxyl, lower alkyl, lower alkoxy, halogen, nitro, amino, cyano or thiol, wherein the lower alkyl or alkoxy is optionally substituted with one or more of hydroxyl, halogen, nitro, amino, cyano, thiol, or a 5- or 6-membered aromatic or non-aromatic ring, optionally substituted with one or more of hydroxyl, lower alkyl, lower alkoxy, halogen, nitro, amino, cyano or thiol; or
Y1 is a 5- or 6-membered aromatic or non-aromatic ring, optionally substituted with one or more groups selected from hydroxyl, lower alkyl, lower alkoxy, halogen, nitro, amino, cyano, carboxy, or thiol, and Y2 is H, if present; or
Y1 and Y2 together with X form a 5- to 8-membered hydrocarbon ring, optionally substituted with one or more groups selected from hydroxyl, lower alkyl, lower alkoxy, halogen, nitro, amino, cyano, carboxyl, or thiol;
Z is a 6-membered heterocyclic or non-heterocyclic ring, one or more groups selected from hydroxyl, lower alkyl, lower alkoxy, halogen, nitro, amino, cyano, carboxyl, or thiol;
R₁ to R₄ are each independently H, hydroxyl, halogen, lower alkyl or lower alkoxy;
with the proviso that the compound is not pentamidine.

21. The method of claim 20, wherein the pentamidine analog has the general Formula (1b) in which Z is phenyl, X is N and Y1 is H.

22. The method of claim 20, wherein the pentamidine analog is administered in conjunction with a bicarbonate adjuvant.

23. The method of claim 22, wherein the bicarbonate is administered at a concentration of about 1 mM to about 900 mM.
